(19) United States
(12) Patent Application Publication
(10) Pub. No.: US 2021/0177462 A1
(43) Pub. Date: Jun. 17, 2021

(54) CERVICAL CERCLAGE APPLICATOR AND METHOD

(71) Applicant: Rapid Stitch LLC, Providence, RI (US)

(72) Inventors: Alexis C. Gimovsky, Providence, RI (US); Felek Jachimowicz, Brookline, MA (US); Ehsan Hajiesmaili, Boston, MA (US); Jordon T. Levine, Providence, RI (US); Gregory A. Walters, McLean, VA (US)

(21) Appl. No.: 17/120,116

(22) Filed: Dec. 12, 2020

Related U.S. Application Data
(60) Provisional application No. 62/947,033, filed on Dec. 12, 2019.

(51) Int. Cl. A61B 17/42; A61B 17/12

(52) U.S. Cl. CPC A61B 17/42; A61B 2017/12018; A61B 2017/4225; A61B 17/12013

(57) ABSTRACT

A device and method for application of a cervical cerclage suture to a patient's cervix is disclosed. The device includes a housing having a first end and a second end spaced from the first end and defining an internal passageway. The device includes a retractable claw having a shaft slidable in the passageway and comprising a plurality of spaced flexible fingers that hold and release the cerclage suture. Each flexible finger may have a through-hole for receiving the cerclage suture, and may have a radial slit in communication with the through-hole and through which the cerclage suture is releasable from each finger. The claw is movable in the housing between a retracted state and an extended state relative to the housing. The shaft is operatively connected to the claw to move the claw axially relative to the housing.

### CROSS REFERENCE TO RELATED APPLICATION

The present application claims the benefit of U.S. Provisional Application No. 62/947,033, filed Dec. 12, 2019, which is incorporated herein by reference in its entirety for all purposes.

### TECHNICAL FIELD

This application is related to a device for application of a cervical cerclage to the cervix of a patient.

### BACKGROUND

Physical and biochemical changes of a human cervix occur physiologically during pregnancy. Cervical remodeling and softening occurs in a gradual manner throughout gestation, and in the final weeks of pregnancy, effacement occurs, increasing the diameter of the cervix and changing the consistency of the cervical canal at the level of the internal os. As the patient enters labor, uterine contractions cause the cervix to further dilate, allowing for the passage of the fetus, and eventually, delivery.

Cervical insufficiency can arise when a cervix is structurally too soft or too short (e.g., 25 mm or less) to remain closed during the entire term of a pregnancy, and can result in a preterm birth and possibly the loss of the fetus. Cervical insufficiency has long been recognized as a potential cause of both spontaneous preterm delivery and second trimester loss. Cervical dilation is often painless, and may go undetected and can also lead to recurrent second trimester pregnancy loss or preterm birth in otherwise healthy women.

Preterm birth is the most common cause of global infant death and is also the leading cause of long-term disability in children. About 15 million babies deliver prematurely each year (5% to 18% of all deliveries) worldwide. Approximately 0.5% of births are periviable births, and these account for the majority of the deaths. A precise cause of a premature birth is not known; the cause of 50% of preterm births is never determined and likely multifactorial. However, it is well known that a history of cervical insufficiency and an incidental short cervix in a pregnant woman are two major risk factors for spontaneous preterm birth.

The relative risk of a short cervix for a spontaneous preterm birth in a pregnant woman is estimated at 2.9 (95% confidence interval 2.1-3.9). Additional risk factors include a prior preterm birth, ethnicity, smoking, obesity and infection during pregnancy. According to statistics provided by the Mayo Clinic, cervical insufficiency in the United States occurs in 1-2% of all pregnancies, but it is thought to cause as many as 20-25% of spontaneous preterm births in the second trimester (the total number of births in US hospitals in 2014 amounted to 3.8 million).

When the cervix starts to shorten and dilate prematurely during a pregnancy (e.g., see FIG. 11A), the above conditions can cause either a second trimester loss or preterm birth. The cervical cerclage, also known as a “cervical stitch”, is a treatment for history of cervical insufficiency, for a short cervix in a patient with a history of prior spontaneous preterm birth or for a patient with a prematurely dilated cervix. The word “cerclage” means “hoop” in French, as in the metal hoop encircling a barrel. Cervical cerclage is a surgical procedure involving a suture sewn into and around the cervix early in the pregnancy, usually performed between weeks 12 to 23 (first or second trimester), and then removed towards the end of the pregnancy (usually 36 weeks) when the greatest risk of miscarriage or preterm birth has passed. The objective of a cerclage procedure is to narrow and mechanically support the cervical canal to prevent preterm labor and premature birth. A cerclage is typically placed as high as possible on the cervix near the cervicovaginal junction. It is estimated that out of 131 million births/year worldwide, there are about 2 million women undergoing cervical cerclage procedures annually.

The procedure is performed under regional or general anesthesia due to pain caused during the placement and application of the cerclage and it is typically performed by an Obstetrician Gynecologist (OBGYN) or a Maternal Fetal Medicine (MFM) subspecialist. The requirement to use anesthesia necessitates the use of the operating room at a monetary cost to the hospital and a cost of time to the physician. It also increases the risk for anesthetic complications in comparison to procedures where anesthesia is not required.

Another approach for treatment of cervical insufficiency is the use of a pessary, which is a ring-like device inserted transvaginally and positioned around the cervix in an effort to reduce direct pressure on the cervical canal by the fetus. However, the efficacy of pessaries in reducing preterm births is difficult to quantify, controversial and still being researched. See, for example, Cervical Pessary for Preventing Preterm Birth in Singleton Pregnancies With Short Cervical Length: A Systematic Review and Meta-analysis. J Ultrasound Med. 2017 August; 36(8):1535-1543.

It therefore would be desirable to improve upon the constraint that known cerclage surgical procedures must be performed in the operating room by developing a rapid, surgery-free, transvaginal cervical cerclage, which may enable this procedure to be executed within the outpatient office setting without general or regional anesthesia.

It also would be desirable to prevent or minimize the occurrence of preterm birth by mechanically assisting the cervical tissue to prevent the inter os of the cervix from opening in a minimally invasive manner.

### SUMMARY

Problems of the prior art have been addressed by implementations disclosed herein, which are based on the use of a cerclage suture (also referenced throughout this disclosure simply as a cerclage or a suture, and also may be referenced as a friction locking member) which includes one or more types of sutures to avoid slippage and based on a unique simple, single-use (e.g., disposable) applicator for placing the cerclage suture around the cervix of a patient in need thereof that is easy to use and simple to manufacture. The device and method in accordance with implementations disclosed herein are designed to adapt to any angle, size, shape, and/or consistency of the cervix, so that the patient's parity should not interfere with the application of the cerclage suture to the cervix, and provide for efficiently completing the cerclage procedure. For purposes of this disclosure, the term cerclage means and refers to the use of a ring or loop to encircle the opening of an insufficient, short or dilated cervix.

This approach is significantly different from any existing procedure developed to prevent preterm birth, including the pessary. The pessary is purported to exert its effect on the cervix by mechanically changing the angle of the cervix relative to the uterus and vagina, and not influencing the consistency or strength of the cervix. Furthermore, the pessary is of controversial efficacy, as it has not been proven to prevent preterm delivery in several clinical trials.

In certain implementations, the device and method are designed to add mechanical strength to or reinforce the cervix and thereby maintain the pregnancy. In certain implementations, the device and method are designed to narrow the cervical opening and thereby maintain the pregnancy. The device has the potential to reduce the number of invasive procedures by approximately 2 million women undergoing this surgical procedure worldwide, which will reduce the risks of surgery including bleeding, infection and ruptured membranes, and also reduce the complications related to anesthesia. The procedure may add approximately 48,000-96,000 hours of Maternal Fetal Medicine (MFM) physician capacity to the United States healthcare system. The procedure may also improve the capacity of the OBGYNs that perform the procedures.

In certain implementations, a friction locking member such as a cerclage suture is manually or robotically deployed with the use of an applicator, and is used in order to avoid slippage of the device relative to the cervix. The applicator is a simple, single-use instrument that is easy to use and relatively low cost to manufacture.

In various implementations, the applicator may include certain components shown in FIG. 1, including a retractable claw comprising a plurality of flexible fingers that are configured with a defined structure at the end of each finger to hold a friction locking member such as a cerclage suture with an untightened knot; a narrow housing that encloses the claw; and a shaft that is operatively connected to the claw to pull and push the retractable claw in and out of the housing. In some implementations, the claw is flexible and retractable, and the flexible fingers of the claw are at first retracted into the housing so that the applicator can be easily inserted into the vagina of a patient. The claw can be pushed out of the housing such that the fingers extend beyond the housing, whereby the fingers of the claw open so that they can be positioned around the cervix of a patient, after which the friction locking member is released from the claw and secured around the cervix. The deployment and/or retraction of the claw can be carried out manually or robotically.

In various implementations, the claw holds the cerclage suture to position it around the cervix and releases it when the ends of the suture are actuated such as by a pulling force. The suture may be threaded through small through-holes on each flexible finger of the claw. These through-holes may communicate with a slit in each finger that is smaller than the thickness of the suture. When the ends of the suture are pulled, the suture is pressed against the slit, and if the pressure is sufficient for the suture to cause the slit to open, the suture will slide out of the slit and be released from the claw. Simultaneously or thereafter, the suture may be tightened about the cervix. To facilitate placement of the cerclage suture around the cervix and tightening of a knot in the suture, a conventional speculum first may be inserted into the vagina and is opened. Then through the speculum, the applicator may be inserted into the vagina and positioned near the cervix. Next, the retractable claw is pushed out of the housing of the applicator and is positioned around the circumference of the cervix, and the end or ends of the suture are pulled which releases the suture from the fingers of the claw and tightens a knot, where present. Subsequently, the claw and fingers are retracted into the housing, and the applicator is removed from the vagina. The end or ends of the suture material may be cut using a pair of scissors or the like and the speculum is removed.

In various implementations, the cerclage is applied without requiring any suturing into the cervix tissue or other tissue of the patient.

According to one innovative aspect of the subject matter described in this application, a device for application of a cervical cerclage to the cervix of a patient includes a housing having a first end and a second end spaced from the first end, the housing defining an internal passageway. The device includes a retractable claw having a shaft slidable in the internal passageway of the housing. The retractable claw includes a plurality of spaced flexible fingers configured to hold and release the cerclage suture. In some aspects, each of the flexible fingers has a through-hole for receiving the cerclage suture. In some aspects, each of the flexible fingers has a radial slit in communication with the through-hole and through which the cerclage suture is releasable from each finger. In some aspects, the claw is movable in the housing between a retracted state in which each of the flexible fingers of the plurality of flexible fingers is at least partially disposed in the passageway and an extended state in which each of the flexible fingers of the plurality of flexible fingers is not disposed in the passageway and extends beyond it. In some aspects, the housing is generally cylindrical. In some aspects, the flexible fingers of the plurality of flexible fingers are spaced apart when the claw is in the extended state. In some aspects, the shaft is operatively connected to the claw to move the claw axially relative to the housing. In some aspects, the cervical cerclage is a barbed suture or is a band. In some aspects, the cervical cerclage is a shape-memory polymer (e.g., biodegradable shape-memory thermoplastic) that shrinks at body temperature.

According to another innovative aspect of the subject matter described in this application, a method of applying a cerclage to the cervix of a patient is disclosed. The method includes introducing into a vagina of a patient a distal end of an applicator, the applicator including a housing having a first end and a second end spaced from the first end, the housing defining an internal passageway. A retractable claw includes a shaft slidable in the internal passageway of the housing. The retractable claw includes a plurality of spaced flexible fingers holding the cerclage suture. The method includes manipulating the applicator to position the cerclage suture about the circumference of the cervix, releasing the suture from the plurality of spaced flexible fingers and tightening the suture about the cervix, and removing the applicator from the patient. In various implementations, the cerclage suture is positioned as high up on the cervix as possible, near the cervicovaginal junction which is also close to the cervical internal os. In certain aspects, the claw is movable in the housing between a retracted state in which each of the flexible fingers of the plurality of flexible fingers is at least partially disposed in the passageway and an extended state in which each of the flexible fingers of the plurality of flexible fingers is not disposed in the passageway and extends beyond it, and wherein the step of manipulating the applicator comprises moving the claw from the retracted state to the extended state, or the step of retracting the claw into the housing prior to the step of removing the applicator from the patient. In some implementations, the claw is not deployed from its retracted position in the housing until it is positioned near or next to the cervical external os of the patient.

In some implementations, a patient in need of the application of a cerclage using the applicator device disclosed herein is a patient with advanced effacement and dilation of the cervix. In some implementations, a patient in need of the application of a cerclage using the applicator device disclosed herein is a patient with clinically diagnosed cervical insufficiency. In some implementations, such clinically diagnosed cervical insufficiency is characterized by a cervical length less than 2.5 cm before 24 weeks of pregnancy. In some implementations, such clinically diagnosed cervical insufficiency is characterized by a prematurely dilated cervix before 24 weeks of pregnancy.

The details of one or more implementations of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

The implementations disclosed herein may take form in various components and arrangements of components, and in various process operations and arrangements of process operations. The drawings are only for purposes of illustrating preferred implementations and are not to be construed as limiting. This disclosure includes the following drawings:

FIG. 1 is an exploded perspective view of individual components of a cerclage applicator in accordance with certain implementations;

FIG. 2 is a perspective view of a cerclage applicator in its assembled and open state in accordance with certain implementations;

FIGS. 3A and 3B are perspective views of a portion of the finger forming part of the claw of the applicator of FIG. 2, and show a cerclage suture positioned in place in the finger, in accordance with certain implementations;

FIG. 4 is a perspective view of the cerclage applicator shown in its retracted state;

FIG. 5 is another perspective view of the cerclage applicator in its assembled and open state;

FIG. 6 is a perspective view of the cerclage applicator highlighting the release feature of the finger supporting a suture in accordance with certain implementations;

FIG. 7 is an exploded perspective view of individual components of a cerclage applicator in accordance with an alternate implementation;

FIG. 8 is a perspective view of the cerclage applicator of FIG. 7 in its assembled and open state;

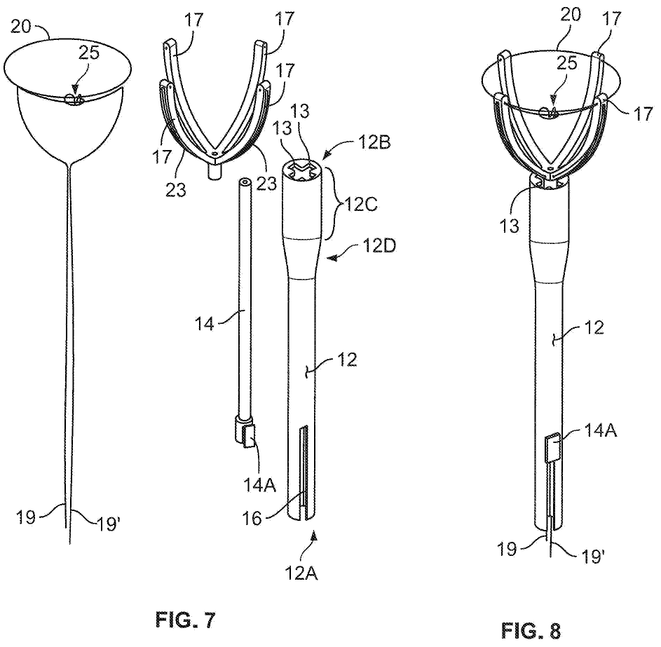

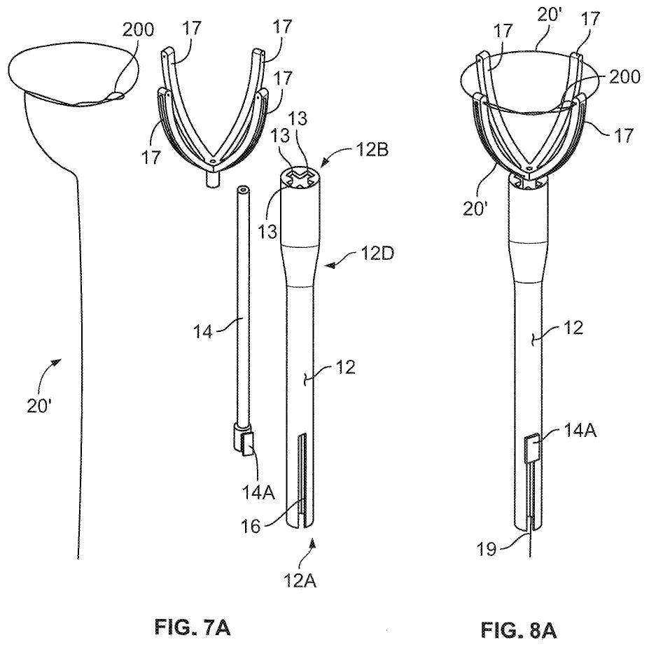

FIG. 7A is an exploded perspective view of individual components of a cerclage applicator with an alternate implementation of a cerclage suture;

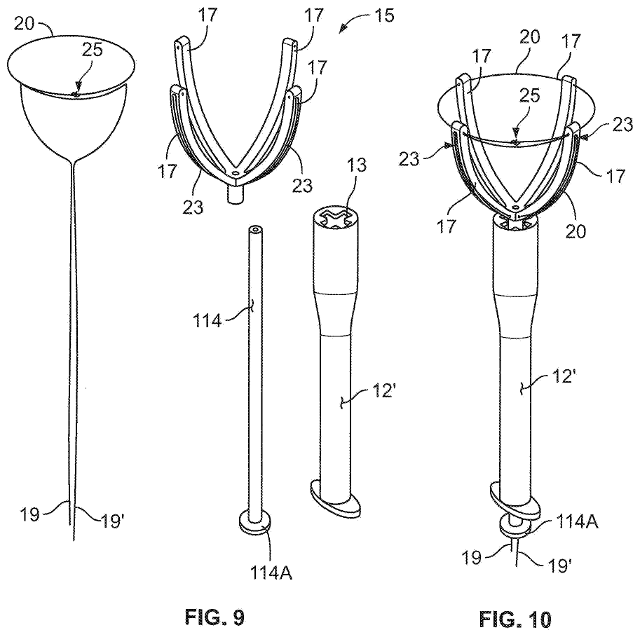

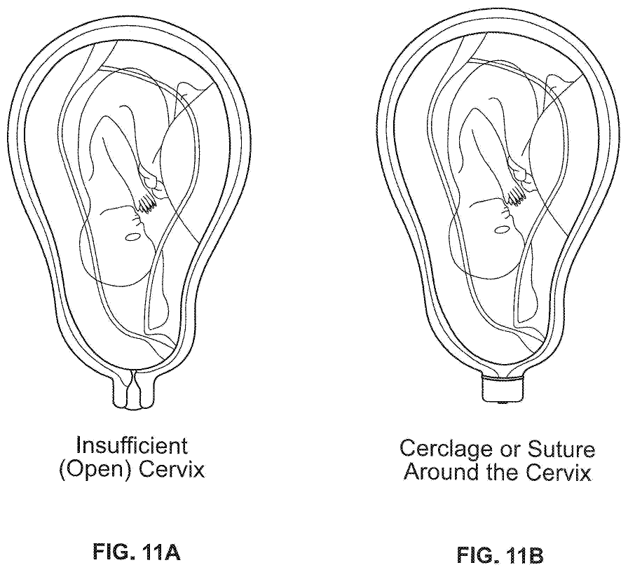

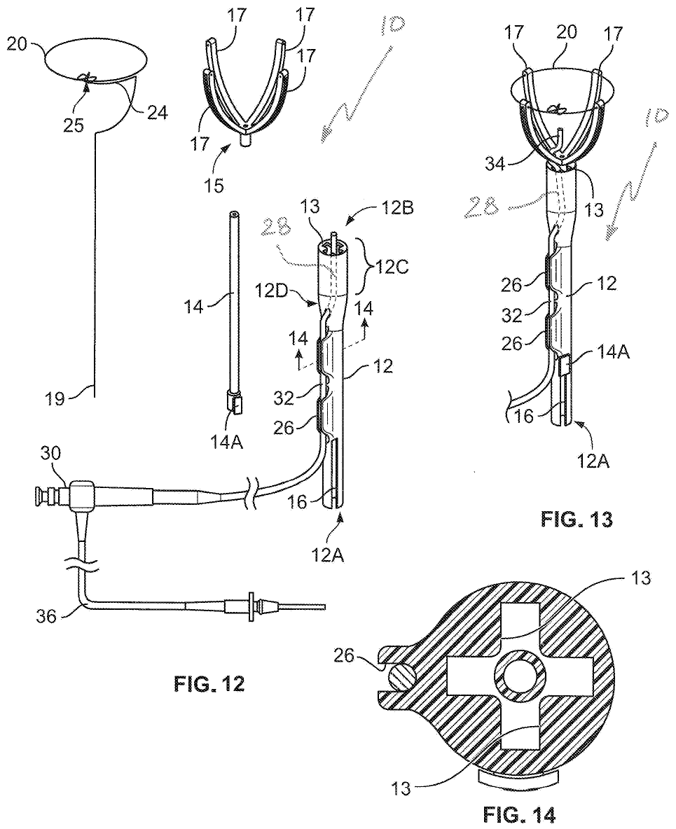

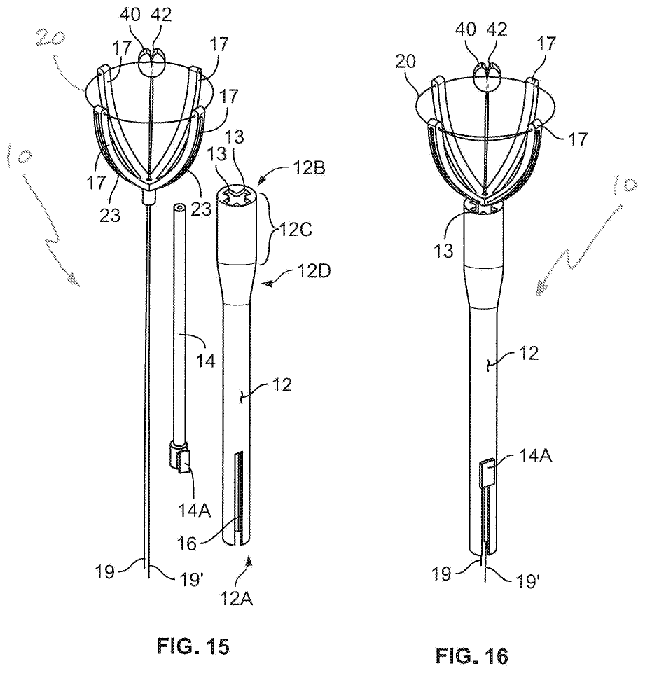

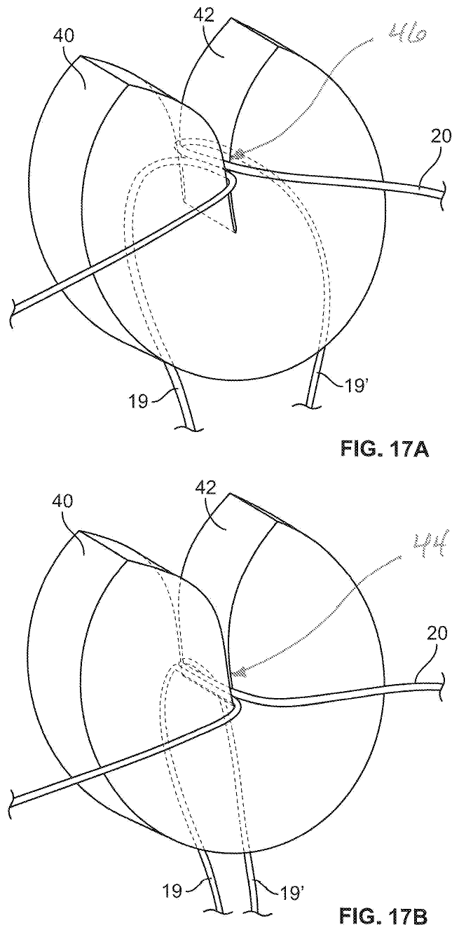

FIG. 8A is a perspective view of the cerclage applicator of FIG. 7A in its assembled and open state;

FIG. 9 is an exploded perspective view of individual components of a cerclage applicator in accordance with another implementation;

FIG. 10 is a perspective view of the cerclage applicator of FIG. 9 in its assembled and open state;

FIG. 11A is an illustration of a cervix with premature shortening and dilation, consistent with cervical insufficiency;

FIG. 11B is an illustration of a cerclage suture positioned around a cervix in an exemplary implementation;

FIG. 12 is an exploded perspective view of individual components of a cerclage applicator configured to accommodate the insertion tube of an endoscope and/or fiber optic light source in accordance with an alternate implementation;

FIG. 13 is a perspective view of the cerclage applicator of FIG. 12 in its assembled and open state in accordance with an alternate implementation;

FIG. 14 is a cross-sectional view taken along line 14-14 of FIG. 12 showing the channel structures configured to accommodate an endoscope and/or fiber optic light source;

FIG. 15 is an exploded perspective view of individual components of a cerclage applicator in accordance with another implementation;

FIG. 16 is a perspective view of the cerclage applicator of FIG. 15 in its assembled and open state;

FIG. 17A illustrates a c-shaped suture anchor clip shown with the sutures in a pre-anchored state; and

FIG. 17B illustrates a c-shaped suture anchor clip shown with the two suture ends in an anchored or locked state.

### DETAILED DESCRIPTION

A more complete understanding of the components, processes and apparatuses disclosed herein can be obtained by reference to the accompanying drawings. The figures are merely schematic representations based on convenience and the ease of demonstrating the present disclosure, and is, therefore, not intended to indicate relative size and dimensions of the devices or components thereof and/or to define or limit the scope of the exemplary implementations.

Although specific terms are used in the following description for the sake of clarity, these terms are intended to refer only to the particular structure of the implementations selected for illustration in the drawing, and are not intended to define or limit the scope of the disclosure. In the drawing and the following description below, it is to be understood that like numeric designations refer to components of like function.

The singular forms “a,” “an,” and “the” include plural referents unless the context clearly dictates otherwise.

As used in the specification, various devices and parts may be described as “comprising” other components. The terms “comprise(s),” “include(s),” “having,” “has,” “can,” “contain(s),” and variants thereof, as used herein, are intended to be open-ended transitional phrases, terms, or words that do not preclude the possibility of additional components.

All ranges disclosed herein are inclusive of the recited endpoint and independently combinable (for example, the range of “from 2 inches to 10 inches” is inclusive of the endpoints, 2 inches and 10 inches, and all the intermediate values).

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. The modifier “about” should also be considered as disclosing the range defined by the absolute values of the two endpoints. For example, the expression “from about 2 to about 4” also discloses the range “from 2 to 4.”

It should be noted that many of the terms used herein are relative terms. For example, the terms “upper” and “lower” are relative to each other in location, i.e. an upper component is located at a higher elevation than a lower component, and should not be construed as requiring a particular orientation or location of the structure. As a further example, the terms “interior”, “exterior”, “inward”, “outward”, “inner” and “outer” are relative to a center, and should not be construed as requiring a particular orientation or location of the structure.

The terms “top” and “bottom” are relative to an absolute reference, i.e. the surface of the earth. Put another way, a top location is always located at a higher elevation than a bottom location, toward the surface of the earth.

The terms “horizontal” and “vertical” are used to indicate direction relative to an absolute reference, i.e. ground level. However, these terms should not be construed to require structures to be absolutely parallel or absolutely perpendicular to each other.

Turning now to FIGS. 1-5, there is shown an implementation of a cervical cerclage applicator device 10 having an elongated housing 12 having a first or proximal end 12A and a second or distal end 12B. The housing 12 defines an outer shell having an interior axial bore or passageway configured to receive an elongated rod or shaft 14, and at least a portion of claw 15 when the claw 15 is in its retracted state. Suitable materials of construction for the housing 12 are plastics including FDA approved plastics for medical devices, such as butadiene styrene (ABS), polyethylenes (LDPE, HDPE), polypropylene, polyethylene terphthalate polyester, and nylon. Acrylonitrile also may be suitable. In certain implementations, the housing 12 has a region 12C of expanded diameter, near the distal end 12B, to accommodate the claw 15 when in the retracted state. In certain implementations, the housing has a region 12D that tapers as it transitions in diameter from the expanded diameter region 12C to a region of smaller diameter, which latter region may be of constant diameter. In other implementations, the entire housing may be of constant diameter, provided the diameter is sufficient to accommodate at least a portion of the claw 15 to enable its partial or complete retraction into the housing 12.

The housing 12 may include a slot 16 that opens at the proximal end 12A. The slot 16 may be configured to accommodate a tab 14A at or near the proximal end of the shaft 14. In some implementations, tab 14A is axially slidable in slot 16 by the surgeon or user of the device, or robotically, to actuate the claw 15 (e.g., to retract or extend the claw 15 relative to the housing 12). Accordingly, the slot 16 should be of the appropriate axial length to enable the shaft to translate a sufficient distance in the housing 12 so that the claw may be retracted into and fully extended from the housing 12.

Alternatively, a syringe-like member 114 may be used as shown in FIGS. 9 and 10. In this implementation, the syringe-like member 114 has a disc-shaped flange 114A at the proximal end which facilitates actuation of the member 114 axially in the elongated housing 12′ to actuate the claw 15 (e.g., to retract or extend the claw 15 relative to the housing 12′). In this implementation, no slot 16 is necessary in the housing 12′. The cerclage suture 20 shown in this implementation is the same configuration as the implementation of FIGS. 7 and 8, but the implementation of the cerclage suture 20′ shown in FIGS. 7A and 8A also may be used.

In some implementations, the distal end of the housing 12 has indents 13 to accommodate the fingers 17 when the claw is retracted. Preferably the number of indents 13 is the same as the number of fingers 17. In the implementation shown, there are four fingers 17 and four indents 13, equally spaced and symmetrical (e.g., they form a “+” shape when viewed from the top or distal end of the housing 12). The indents 13 may extend axially the depth of expanded diameter region 12C. In some implementations, those skilled in the art will appreciate that fewer or more than four fingers 17 may be used.

In certain implementations, shaft 14 (and shaft 114 in the FIG. 9 implementation) is a generally cylindrical elongated member and has an outer diameter smaller than the smallest internal diameter of the housing 12, so that the shaft 14 fits in the internal passageway in the housing 12 and is translatable, such as by sliding, therein. The shaft 14 may be made of an FDA approved plastic for medical devices mentioned above. The shaft 14 may be formed with an integral claw 15, or the claw 15 may be fixedly mounted or attached to the shaft by any suitable means, including with an adhesive, a fastener such as a pin, or by ultrasonic welding. Preferably the shaft 14 has an internal passage of sufficient diameter to receive a tether 19, such as a tether to cerclage suture 20 as discussed in greater detail below.

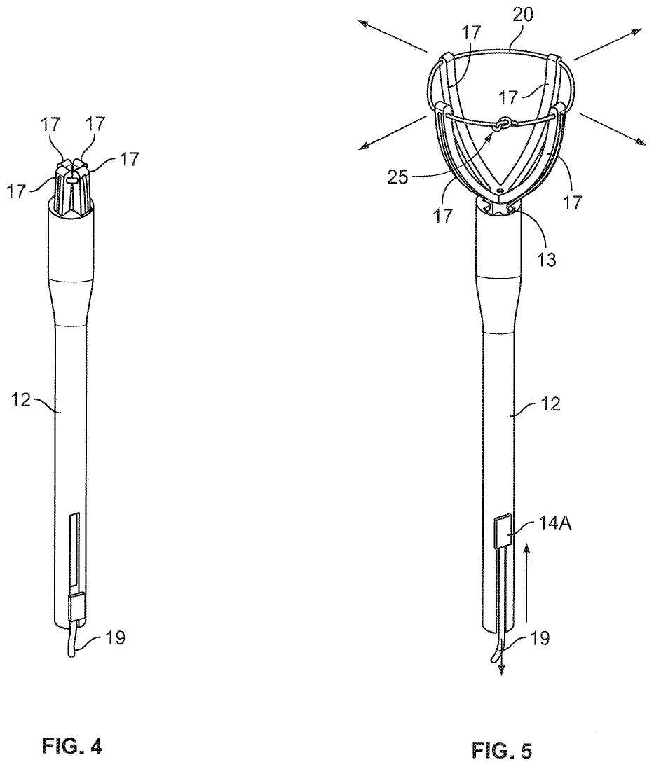

As best seen in FIGS. 4 and 5, in certain implementations the claw 15 includes a plurality of flexible fingers 17. The flexibility of the fingers 17 allows for them to move or collapse radially towards each other as the claw 15 is retracted into the housing 12. In the retracted state as shown in FIG. 4, each of the fingers 17 is received by a respective one of the indents 13 in the housing 12, and is substantial axial alignment with the housing 12. Suitable materials for the flexible fingers 17 include silicone (polydimethylsiloxane) and polyurethane. In the implementation shown, the fingers 17 do not retract completely into the housing 12; they partially extend out of the housing. In other implementations, the fingers 17 can retract completely into the housing 12 with no portion exposed. In some implementations, in the retracted state, the fingers contact one another, and have an overall reduced diameter compared to their expanded state, facilitating insertion of the device into the vagina of a patient.

Actuation of the device to attach the suture 20 to the cervix of a patient forces the fingers 17 out of the housing, thereby spreading the fingers 17 from their contracted state in a direction away from each other as shown by the arrows in FIG. 5, increasing their radial profile. In the implementation shown in FIG. 5, the claw 15 is in its fully expanded state, with the fingers 17 in their normally biased spread configuration. In certain implementations, the fingers 17 are shaped, dimensioned and configured such that when in the expanded state, they may be cooperatively positioned around the exterior of the cervix of a patient. In other words, the internal region defined by the fingers 17, when the claw 15 is in its fully expanded state, is shaped to conform to and receive a cervix of a patient.

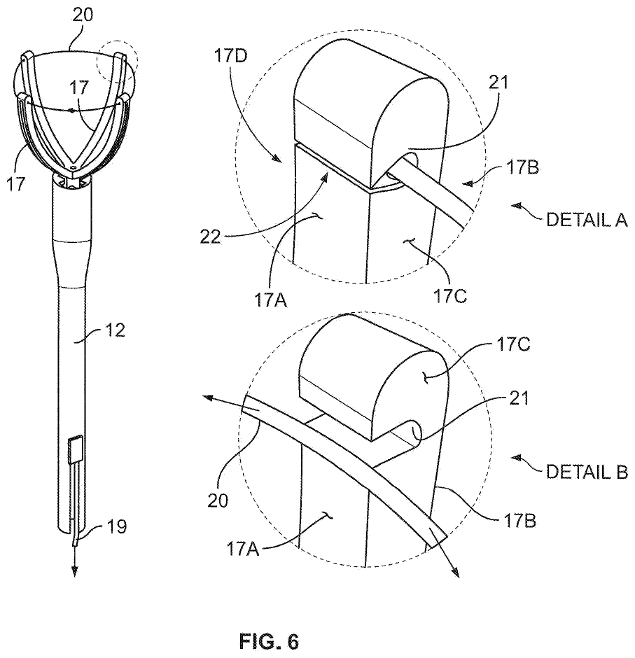

Turning now to FIGS. 3A, 3B and 6, details of an exemplary flexible finger 17 in accordance with certain implementations are shown. In the implementation shown, each finger 17 has a leading edge 27 that may be rounded for ease of insertion into the patient. In some implementations, each flexible finger 17 has an inner face 17A, an opposite outer face 17B, and two side faces 17C and 17D opposite each other. Each finger 17 includes a through-hole 21 extending between the two side faces 17C, 17D as shown. The through-hole 21 may communicate with a transverse slit 22 formed in the inner face 17A. In view of the resiliency of the material used for the fingers 17, the height of the slit 22 is relatively small, preferably smaller than the diameter or thickness of the suture 20, so that the suture 20 (and sutures 20 and 24 in FIG. 3B) occupies the region defined by the through-hole 21 and does not normally release from the finger 17 through the slit 22, as shown in Detail A of FIG. 6. Upon the application of sufficient force to the suture 20, the suture 20 is caused to move towards the slit 22 and force the slit to open further, as shown in Detail B of FIG. 6.

In some implementations, at least one of the fingers may have a longitudinal groove 23 formed on the outer face 17B as best seen in FIGS. 3A, 3B and 10. The groove 23 communicates with through-hole 21, allowing the suture 20 to be threaded into the groove 23 as shown. This portion of the suture 20 that is received by the groove may be elongated and extend into the interior passageway of the housing 12, and extend out the proximal end 12A of the housing 12, defining tether 19, as best seen in FIGS. 4 and 5. The tether is accessible for the user to create a pulling force to release the suture 20 from the claw 15.

In the implementations of FIGS. 7 and 9, there may be two longitudinal grooves 23, one each in adjacent fingers 17, and two tethers 19 and 19′. FIGS. 7 and 8 show an implementation similar to that of FIG. 1, except that two tethers 19, 19′ are present.

In certain implementations, in the assembled device, the suture 20 is threaded through each of the through-holes 21 of each finger 17, through groove 23, and down the passageway of the housing 12. In view of the cooperative configuration of the fingers 17 in their normal, expanded state as shown in FIG.5, the portion of the suture 20 that is threaded through the through-holes 21 of the fingers assumes a substantially circular configuration as shown. The shape that the suture 20 assumes when positioned in the claw 15 is not particularly limited; it is a function of the relative positioning of the fingers 17, and can be adjusted accordingly so that the suture 20 can be appropriately positioned around the cervix of a patient.

In certain implementations, the suture 20 is a suture having at least one knot 25 formed in portion of it, preferably between two fingers 17. The knot is formed such that upon the application of a pulling force to tether 19 (or to tethers 19 and 19′), the knot 25 tightens from a normally loosened state. As the knot 25 tightens, it causes the suture to move radially inwardly into each of the slits 22, expand the slits 22, and release from the fingers 17. Alternatively or in addition, the tightening of the knot can be used to tighten the suture about the cervix. One suitable knot 25 is a constrictor knot.

In some implementations, the cerclage suture 20 is a suture that has a roughened outer surface, such as a ribbed or barbed surface, to assist it in remaining secure on the cervix once applied. One suitable suture is the V-loc™ wound closure material commercially available from Medtronic. Such an implementation is shown in FIGS. 7A and 8A, where the suture 20′ includes a preformed loop 200 (e.g., a V-loc™ wound closure band) at one end, and the remainder of the suture 20′ may be threaded through the loop 200, similar to a zip tie. One or more of the barbs on the suture 20′ then self-latches to the loop 200. The suture 20′ is then anchored into the tissue of the cervix by the barbs. As shown, the suture 20′ includes a single tether 19 that sits in the passageway of shaft 14 and is accessible for the user (FIG. 8A) to create a pulling force to release the suture 20′ from the claw 15. The V-loc™ wound closure device may have a plurality of barbs on its outer surface which assist in securing the suture to the cervix.

Preferably the cerclage suture 20 is made of a non-absorbable material, such as Nylon, polypropylene, polyester, prolene or ethibond, etc. Another suitable cerclage suture 20 is a MERSILENE□ polyester fiber suture made of polyethylene terephthalate, commercially available from Johnson & Johnson. In some implementations, the cerclage suture 20 may be a band.

In some implementations, the cerclage suture 20 is a shape-memory polymer suture that shrinks. The shape-memory polymer can have a knot that tightens when shrunk or can have a closed loop shape that tightens around the cervix when shrunk. It comprises a band that is an elastic material that stretches around the cervix and then subsequently self-cinches around the exterior of the cervix.

In various implementations, the claw holds the cerclage suture 20 or 20′ to position it around the cervix and releases the suture 20 when the tether 19 or tethers 19, 19′ of the suture 20 are pulled. The suture 20 (or band 200) is threaded through small through-holes 21 on each flexible finger 17 of the claw. These through-holes 21 communicate with a slit 22 that is smaller than the thickness of the suture 20, and opens to the interior face of the finger 17. When the tether or tethers of the suture 20 or 20′ are pulled, the suture 20 (or band 200) is forced against each slit 23 and if the pressure is sufficient for the slit 23 to open to an extent greater than the thickness or diameter of the suture 20, the suture 20 (or band 200) will slide out of each slit 23 and be released from the claw so that it sits around the cervix. Simultaneously, the suture 20 is tightened around the cervix.

To place the cerclage suture 20 around the cervix and tighten the knot 25, a conventional speculum (not shown) may be inserted into the vagina and is opened, to expand the vaginal opening. Then through the speculum, the distal end of the applicator is inserted into the vagina and placed in operative position around the cervix, e.g., it is inserted to an extent such that the distal end is up to or next to the cervical external os of the patient. Next, the retractable claw 15 is deployed by pushing it out of the housing 12 and is positioned around the cervix. A cerclage is typically placed as high as possible on the cervix near the cervicovaginal junction. The tether 19 or tethers 19, 19′ of the suture 20 are pulled, which releases the suture 20 from the fingers 17 of the claw and tightens the knot 25, securing the suture around the cervix, as illustrated in FIG. 11B. The end of suture may be cut, such as with a pair of scissors. Subsequently, the claw 15 may be retracted into the housing, the applicator may be removed from the vagina, and the speculum may be removed. A similar procedure applies for suture 20′.

Although the foregoing description involves manual insertion of the applicator and manual deployment and retraction of the claw 15, those skilled in the art will appreciate that one or more of the manual operations may be carried out robotically.

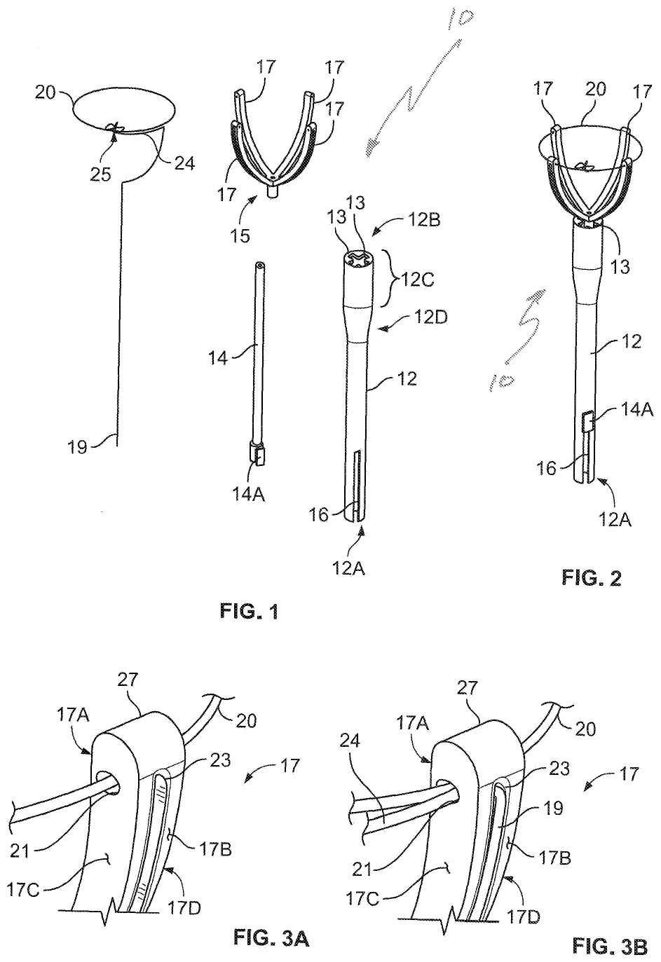

Turning to FIGS. 12 and 13, an alternate implementation of the cervical cerclage applicator device 10 is shown for use in combination with an endoscope 30 that may be used for visualization and/or as a light source for placing the cerclage suture 20 and examining the cervix. The device 10 illustrated in FIGS. 12 and 13 is substantially similar to that shown in FIGS. 1 and 2, and the description for the reference numbers in FIGS. 1 and 2 describes the same features. FIGS. 12 and 13 show that the housing 12 includes two channel structures 26 that are formed or molded at different points along the length of the housing 12. The channel structures are dimensioned to accommodate and securely retain the insertion end 32 of a suitable endoscope 30.

As shown, the housing 12 can also be formed or molded to include an internal passage 28 to securely hold the insertion end 32 of the endoscope 30. The end portion 34 can be inserted through the internal passage 28 and extend up to the top portion of the housing 12, and depending on the particular circumstances of the procedure, the end portion 34 may extend a short distance beyond the top portion of the housing 12. In a preferred implementation, the end portion 34 emits light transmitted through the endoscope 30, or from a suitable light source for illuminating the placement area for the cerclage suture 20.

The endoscope 30 as shown also may include a tube 36 to carry the light that is then transmitted through the endoscope 30, through the insertion end 32, and emitted through the end portion 34. The tube 36 includes a connection end that plugs into a suitable light source, not specifically shown, or may be a stand-alone light source that does not require a plug. In one exemplary implementation, the light source may be a small battery powered LED light that is secured to region 12C of the housing 12, or inserted into the internal passage 28. In another exemplary implementation, the small batter powered LED light may include an adhesive backing such that when a thin protective cover is peeled away from the adhesive backing, the adhesive surface is exposed and the battery is connected to the LED to produce a light source. The adhesive surface side is then applied against the outer surface of the housing 12 around region 12C.

FIG. 14 is a cross-sectional view of the housing 12 taken along line 14-14 of FIG. 12 and illustrates the channel structure 26 with the insertion end 32 of the endoscope 30 fully seated within the channel structure 26. As shown, each of the channel structures 26 are formed by two parallel wings that protrude from the housing 12. The space between the wings is appropriately dimensioned so that the insertion end 32 of the endoscope 30 can be easily and quickly snap fitted into the channel structure 26, and also allows the insertion end 32 to be easily separated from the housing 12 once the cerclage procedure is completed.

A particular advantage provided by the channel structure 26 and the internal passage 28 is that an endoscope 30 can be securely attached to the housing 12 for use with the applicator device 10, and as the device is guided for placement of the cerclage suture 20, the lighted end 34 of the endoscope 30 is guided along with the device 10 to illuminate the placement area for the suture 20.

Another advantage of this arrangement is that the physician performing the procedure can decide whether or not the extra light and/or visualization provided by the endoscope 30 will assist with placing and securing the cerclage suture 20 during the procedure. If its determined that extra light will assist, even at the time of performing the procedure, the pre-sterilized lighted end portion 34 can be quickly threaded through the internal passage 28, and the insertion end 32 of the endoscope 30 can be attached to the housing 12 with minimal effort and no additional components. Once the cerclage procedure is completed, the insertion end 32 can be quickly separated from the housing 12 and the device 10 can be properly discarded.

With reference to FIGS. 15 and 16, an alternate implementation of the cerclage applicator device 10 is shown for use in connection with a c-shaped suture anchor clip 40 where the two ends of the suture 20 are secured in a v-shaped notch 42 formed into the body of the suture anchor clip 40. The individual components of the cerclage applicator device 10 shown in FIGS. 15 and 16 are substantially similar to those described with respect to FIGS. 7 and 8.

As described with the previous implementations, the cerclage suture 20 is threaded through each of the through-holes 21 of each finger 17. In view of the cooperative configuration of the fingers 17 in their normal, expanded state, the portion of the suture 20 that is threaded through the through-holes 21 of the fingers 17 assumes a substantially circular configuration as shown. The shape that the suture 20 assumes when positioned in the claw 15 is not particularly limited; it is a function of the relative positioning of the fingers 17, and can be adjusted accordingly so that the suture 20 can be appropriately positioned around the cervix of a patient.

However, in the implementation shown in FIGS. 15 and 16, the free ends of the suture 20 are secured with a c-shaped suture anchor clip 40 having a v-shaped notch 42 for receiving the free ends of the suture 20. The tether ends 19, 19′ are then threaded through a hole formed in the bottom of the claw 15. For final assembly of the applicator device 10, the tether ends 19, 19′ are threaded through the internal passage of the shaft 14 and may extend beyond the lower end of the housing 12. Alternatively, the tethers 19, 19′ may be positioned along the side of the claw 15 and run down through one of the indents 13, down alongside the shaft 14, and extend downward through the space between the shaft 14 and the internal passageway in the housing 12, and may extend beyond the lower end of the housing 12.

FIGS. 17A and 17B illustrate an exemplary c-shaped suture anchor clip 40 that may be used with the alternate implementation of the cerclage applicator device 10 shown in FIGS. 15 and 16. It should be understood that while c-shaped suture anchor clips are shown that other types of suture anchor clips are suitable. For example, the suture anchor clips may be u-shaped or a modified u-shape. In some implementations the suture anchor clip is compressed around the suture threads using an instrument designed for this purpose for permanently securing the ends of the suture 20. The suture anchor clip 40 is formed to include a v-shaped notch 42 that tapers down to a narrow channel 44 in the bottom portion of the v-shaped notch 42. The dimensions of the v-shaped notch 42, and specifically the narrow channel 44, are appropriately sized to secure the free ends of the suture 20, and to prevent the free ends from moving or dislodging from the narrow channel 44 after the suture 20 is properly secured around the cervix. In a preferred implementation, the suture anchor clip is formed, molded and/or machined from a suitable medical grade plastic, and may be formed in a contrasting color for easy visualization during the placement procedure.

FIG. 17A illustrates the ends of the suture 20 placed within a mid-portion 46 of the v-shaped notch 42, prior to placement of the cerclage suture 20, such that the dimensions of the mid-portion 46 will allow for adjustment and appropriate tightening of the suture 20 during the placement procedure before permanently securing the suture 20. Once the suture 20 is placed and the free ends of the suture 20 are appropriately tightened, FIG. 17B illustrates that the free ends of the suture 20 are wedged into the narrow channel 44 to lock them into place. Once the cerclage suture 20 is placed and the free ends are secured, the tethers 19, 19′ are trimmed with scissors.

The v-shaped notch 42 may optionally include internal structure formed within the notch 42, and more specifically within the mid-portion 46 that allow the tethers 19 and 19′ to be pulled only in one direction to tighten to cerclage suture 20 around the cervix once properly placed, but the internal structure at the mid-portion 46 is formed such that the suture threads 20 are prevented from moving in the opposite direction, thus creating a one-way locking feature to prevent the cerclage suture 20 from coming loose through the steps of the placement procedure.

As illustrated in FIG. 17B, an additional advantage of the v-shaped notch 42 is that once the cerclage suture 20 is properly placed and the tethers 19, 19′ are appropriately tightened, the suture threads can be wedged into the lowest portion of the narrow channel 44 to secure the suture threads and prevent the cerclage 20 from coming loose.

While various aspects and implementations have been disclosed herein, other aspects, implementations, modifications and alterations will be apparent to those skilled in the art upon reading and understanding the preceding detailed description. The various aspects and implementations disclosed herein are for purposes of illustration and are not intended to be limiting. It is intended that the present disclosure be construed as including all such aspects, implementations, modifications and alterations insofar as they come within the scope of the appended claims or the equivalents thereof. Accordingly, other implementations are within the scope of the following claims.

What is claimed is:

1. A device for application of a cervical cerclage to the cervix of a patient, the device comprising:
a housing having a first end and a second end spaced from the first end, the housing defining an internal passageway; and
a retractable claw having a shaft slidable in the internal passageway of the housing; the retractable claw including a plurality of spaced flexible fingers configured to hold and release the cerclage.

2. The device of claim 1, wherein each of the flexible fingers includes a through-hole for receiving the cerclage.

3. The device of claim 1, wherein each of the flexible fingers has a radial slit in communication with the through-hole and through which the cerclage is releasable from each finger.

4. The device of claim 1, wherein the claw is movable in the housing between a retracted state in which each of the flexible fingers of the plurality of flexible fingers is at least partially disposed in the passageway and an extended state in which each of the flexible fingers of the plurality of flexible fingers is not disposed in the passageway and extends beyond it.

5. The device of claim 1, wherein the housing is generally cylindrical.

6. The device of claim 1, wherein the flexible fingers of the plurality of flexible fingers are spaced apart when the claw is in the extended state.

7. The device of claim 1, wherein the shaft is operatively connected to the claw to move the claw axially relative to the housing.

8. The device of claim1, wherein the cerclage is a suture.

9. The device of claim 8, wherein a suture anchor clip secures the suture.

10. The device of claim 1, wherein the cerclage is a barbed suture.

11. The device of claim 1, wherein the cerclage is a band.

12. The device of claim 1, wherein the housing further comprises one or more channel structures defining a channel configured to receive an insertion end of an endoscope, wherein the insertion end of the endoscope allows at least one of visualization for application of the cerclage to the cervix, and a light source for application of the cerclage to the cervix.

13. The device of claim 12, wherein the one or more channel structures are defined by two parallel wings that protrude from the housing, and wherein the two parallel wings are configured to receive the insertion end of the endoscope.

14. The device of claim 1, wherein an internal passage is formed within the housing at an end of the housing proximate to the retractable claw, and wherein the internal passage is configured to receive at least one of an insertion end of an endoscope, and a light source.

15. A method of applying a cerclage to the cervix of a patient, the method comprising:
introducing in a vagina of the patient a distal end of an applicator comprising a housing having a proximal end and the distal end spaced from the proximal end, the housing defining an internal passageway; a retractable claw having a shaft slidable in the internal passageway of the housing; the retractable claw comprising a plurality of spaced flexible fingers holding the cerclage;
manipulating the applicator to position the friction locking member about the cervix;
releasing the friction locking member from the plurality of spaced flexible fingers and tightening the cerclage about the cervix; and
removing the applicator from the patient.

16. The method of claim 15, wherein the claw is movable in the housing between a retracted state in which each of the flexible fingers of the plurality of flexible fingers is at least partially disposed in the passageway and an extended state in which each of the flexible fingers of the plurality of flexible fingers is not disposed in the passageway and extends beyond it, and wherein the step of manipulating the applicator comprises moving the claw from the retracted state to the extended state.

17. The method of claim 15, wherein the claw is movable in the housing between a retracted state in which each of the flexible fingers of the plurality of flexible fingers is at least partially disposed in the passageway and an extended state in which each of the flexible fingers of the plurality of flexible fingers is not disposed in the passageway and extends beyond it; the method further comprising retracting the claw into the housing prior to the step of removing the applicator from the patient.

18. The method of claim 15, wherein the step of manipulating the applicator to move the claw from the retracted state to the extended state is carried out when the distal end of the applicator is next to the cervical external os of the patient.

19. A device for application of a cervical friction locking member to the cervix of a patient, the device comprising:
a housing having a first end and a second end spaced from the first end, the housing defining an internal passageway;
a retractable claw having a shaft slidable in the internal passageway of the housing; the retractable claw comprising a plurality of spaced flexible fingers configured to hold and release the friction locking member; and
a suture anchor clip configured to secure the friction locking member.

20. The device of claim 19 wherein the friction locking member is a suture.
